(19) United States
(12) Patent Application Publication
(10) Pub. No.: US 2025/0302813 A1
(43) Pub. Date: Oct. 2, 2025

(54) RECOMBINANT METHIONINASE IN THE TREATMENT OF CANCER

(71) Applicant: ANTICANCER INC, San Diego, CA (US)

(72) Inventors: BRAD BARSAM CHOOBIN, Irvine, CA (US); YUTARO KUBOTA, San Diego, CA (US); QINGHONG HAN, San Diego, CA (US); SEI MORINAGA, San Diego, CA (US); KOHEI MIZUTA, San Diego, CA (US); ROBERT M. HOFFMAN, San Diego, CA (US)

(21) Appl. No.: 19/097,356

(22) Filed: Apr. 1, 2025

Related U.S. Application Data
(60) Provisional application No. 63/572,771, filed on Apr. 1, 2024.

(51) Int. Cl. A61K 31/44; A61K 38/51; A61P 35/00

(52) U.S. Cl. CPC A61K 31/44; A61K 38/51; A61P 35/00; C12Y 404/01011

(57) ABSTRACT

A combination of recombinant methioninase and regorafenib for the treatment of cancer. The combination of recombinant methioninase and regorafenib shows a synergistic effect that allows for a reduction in the dose of regorafenib, and thus decreases the dose-dependent toxicity of regorafenib.

### CROSS REFERENCE TO RELATED APPLICATIONS

This application claims priority from a U.S. Provisional Patent Appl. No. 63/572,771, filed on Apr. 1, 2024, which is incorporated herein by reference in its entirety.

### FIELD OF INVENTION

The present invention relates to a recombinant methioninase, and more specifically, the present invention relates to a synergistic combination of recombinant methioninase and regorafenib for the treatment of cancer.

### BACKGROUND

Regorafenib is an oral multi-kinase inhibitor that acts by targeting vascular endothelial growth factor receptor 2, fibroblast growth factor receptor 1, and other oncogenic kinases. Regorafenib has a molecular formulae C₂₁H₁₅ClF₄N₄O₃ and IUPAC name 4-[4-[[4-chloro-3-(trifluoromethyl)phenyl]carbamoylamino]-3-fluorophenoxy]-N-methylpyridine-2-carboxamide. Regorafenib has been approved for oral administration in the treatment of advanced forms of cancers, such as metastatic colorectal cancer, advanced gastrointestinal stromal tumors, and advanced hepatocellular carcinoma. In several studies, it was found that regorafenib increased the overall survival of patients with metastatic colorectal cancer and gastrointestinal stromal tumors with whom first-or second-line therapies have failed. However, the toxicity of regorafenib appears within 3-4 days of the start of treatment and includes severe skin rash, diarrhea, hypertension, and fatigue. These severe side effects make it difficult to start regorafenib at the recommended dose of 160 mg/day. Administration of Regorafenib at the dose of 160 mg/day results in increased blood levels of its active metabolites (M2, M5), which are responsible for the severe side effects. Thus, the dose intensity of regorafenib has to be reduced due to its severe side effects.

Recently, clinical trials showed that starting at a low dose of regorafenib (80 or 120 mg/day) can reduce side effects and increase dose intensity. According to these results, the National Comprehensive Cancer Network (NCCN) Clinical Practice Guidelines in Oncology recommends a starting dose of 80 mg/day rather than the usual 160 mg/day dose, with dose escalation depending on side effects. However, lowering the dose of regorafenib also decreases efficacy for some patients.

Thus, a need is appreciated for reducing the dose of Regorafenib without affecting the efficacy of the drug.

### SUMMARY OF THE INVENTION

The following presents a simplified summary of one or more embodiments of the present invention to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to an oral combination of regorafenib and recombinant methioninase (rMETase) for the treatment of cancer.

Another object of the present invention is that the combination has a synergistic effect.

Still another object of the present invention is that the dose of regorafenib can be reduced, thus decreasing the dose-related toxicity of regorafenib.

In one aspect, disclosed is a method for treatment of cancer in a patient suffering from the cancer, the method comprises administering regorafenib, or a hydrate, an isomer, a solvate, a metabolite, a pharmaceutically acceptable salt, or a prodrug thereof, and recombinant methioninase in effective amounts to the patient. The administration is through an oral route. The cancer is metastatic colorectal cancer, advanced gastrointestinal stromal tumors, or advanced hepatocellular carcinoma.

In one aspect, disclosed is a composition for the treatment of cancer, the composition comprises regorafenib, or a hydrate, an isomer, a solvate, a metabolite, a pharmaceutically acceptable salt, or a prodrug thereof; and recombinant methioninase. The composition is for oral administration.

In one aspect, disclosed is an oral dosage form for the treatment of cancer, the oral dosage form comprising regorafenib, or a hydrate, an isomer, a solvate, a metabolite, a pharmaceutically acceptable salt, or a prodrug thereof; recombinant methioninase, and pharmaceutically acceptable excipients. The oral dosage form is a tablet or a capsule.

### BRIEF DESCRIPTION OF DRAWINGS

The accompanying figures, which are incorporated herein, form part of the specification and illustrate embodiments of the present invention. Together with the description, the figures further explain the principles of the present invention and enable a person skilled in the relevant arts to make and use the invention.

FIG. 1 shows the in vitro efficacy of recombinant methioninase (rMETase) on HCT-116 (human colon cancer) cells. Cell viability was measured with the WST-8 reagent. The concentration axis uses a log₂ scale. IC₅₀: Half-maximal inhibitory concentration.

FIG. 2 shows the in vitro efficacy of regorafenib on HCT-116 cells. Cell viability was measured with the WST-8 reagent. The concentration axis uses a log₂ scale. IC₅₀: Half-maximal inhibitory concentration.

FIG. 3 shows the synergistic effect of the combination of recombinant methioninase (rMETase) and regorafenib at their half-maximal inhibitory concentrations (0.61 U/ml and 2.2 μM, respectively) against HCT-116 cells in vitro. Cell viability was measured with the WST-8 reagent.

FIG. 4 shows that recombinant methioninase (rMETase) lowered the half maximal inhibitory concentration (IC₅₀) of regorafenib against HCT-116 cells in vitro from 2.26 μM to 1.46 μM. Cell viability was measured with the WST-8 reagent. The concentration axis uses a log₂ scale. IC₅₀: Half-maximal inhibitory concentration.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is to describe particular embodiments only and is not intended to be limiting of embodiments of the invention. As used herein, the singular forms “a”, “an”, and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention, since the scope of the invention will be best defined by the allowed claims of any resulting patent.

The invention described pertains to a combination of regorafenib and recombinant methioninase (rMETase) for the treatment of cancer. Recombinant methioninase (rMETase) can lower the effective concentration of regorafenib in vitro against colorectal-cancer cells when the two are administered in combination.

Regorafenib is a third-line chemotherapy drug for metastatic colorectal cancer and gastrointestinal stromal tumor, used after the failure of first-or second-line therapy. The problem with regorafenib is its dose-limiting toxicity. The Applicants found that rMETase, when combined with regorafenib, can reduce the effective dose of regorafenib and thereby also reduce its toxicity.

Regorafenib is a multi-kinase inhibitor of angiogenic, stromal, and oncogenic-receptor tyrosine kinases. In an in vitro study using the human colorectal cancer cell line, it was found that regorafenib increased the intracellular S-adenosyl-L-methionine (SAM) level by 34.5% compared to the control (Ogihara S, Metabolic-pathway-oriented screening targeting S-Adenosyl-I-methionine reveals the epigenetic remodeling activities of naturally occurring catechols. J Am Chem Soc. 142(1) 21-26 2020). SAM is the only provider of methyl groups to DNA, RNA, histones, and other proteins. This suggests that regorafenib inhibits transmethylation reactions that use SAM. Methionine depletion reduces SAM levels in cells, further reducing transmethylation reactions, which are elevated in cancer cells. Therefore, the combination of rMETase and regorafenib has a synergistic effect, giving rise to the possibility of lowering the effective dose of regorafenib in patients, thereby reducing its severe toxicity, and increasing the use of regorafenib for the treatment of different types of cancer. Also, rMETase targets methionine addiction, the fundamental hallmark of cancer.

### EXPERIMENTATION

### Materials and Methods

Cell culture: The HCT-116 human colon cancer cell line was acquired from the American Type Culture Collection (Manassas, VA, USA). The cells were grown in Dulbecco's minimum essential medium (DMEM) with 10% fetal bovine serum and 100 IU/ml of penicillin/streptomycin.

rMETase production and formulation: rMETase was produced at AntiCancer Inc. (San Diego, CA, USA) by fermentation of recombinant Escherichia coli transformed with the methioninase gene from Pseudomonas putida. rMETase was purified using a high-yield method, including a 60° C. thermal step, polyethylene-glycol precipitation, and diethylaminoethyl-sepharose fast-flow column chromatography.

Cell viability testing: HCT-116 cells were cultivated at subconfluence overnight in Dulbecco's modified Eagle's medium in 96-well plates (1.0×10³ cells per well). The following day, HCT-116 cells were treated with concentrations of rMETase ranging from 0.125 to 8 U/ml or regorafenib ranging from 0.25 μM to 16 μM. Regorafenib was purchased from MedChemExpress (Monmouth Junction, NJ, USA). After 72 h of treatment, cell viability was assessed using the Cell Counting Kit-8 (Dojindo Laboratory, Kumamoto, Japan) with the WST-8 reagent.

ImageJ version 1.53 (National Institutes of Health, Bethesda, MD, USA) was applied to calculate IC₅₀ values and sensitivity curves. After calculating the half-maximal inhibitory concentration (IC₅₀) for rMETase and regorafenib, the ICs. concentrations of both drugs were used to determine the synergistic efficacy of the combination of the drugs. Finally, the IC₅₀ study was repeated for regorafenib on HCT-116 cells treated in combination with the IC₅₀ concentration of rMETase to examine whether methionine restriction increased the efficacy of regorafenib. Each experiment was carried out in triplicate.

Statistics: GraphPad Prism 9.4.0 (GraphPad Software, Inc., San Diego, CA, USA) was used to conduct all statistical analyses. Tukey's multiple comparison test was performed for the parametric test of comparison between groups. All data are presented as the mean and standard deviation. The significance level was p≤0.05.

### Results

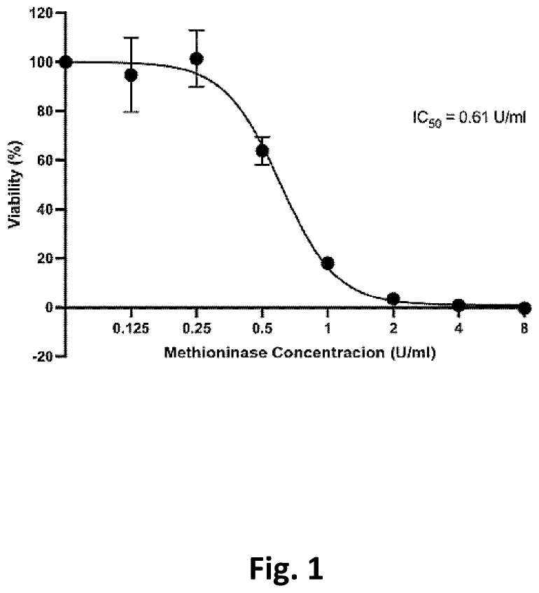

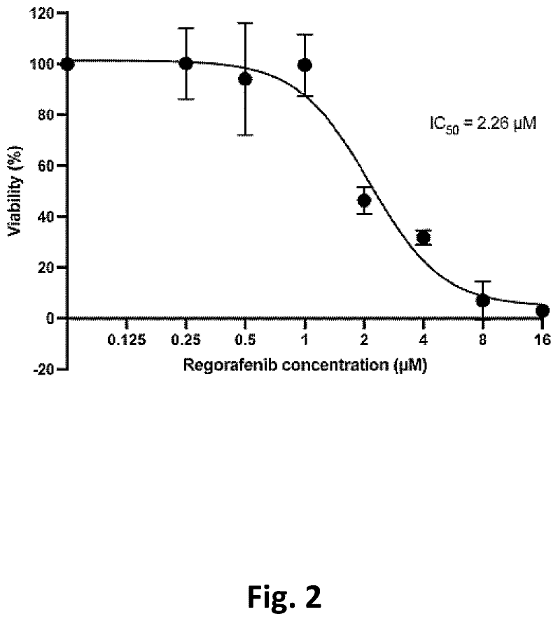

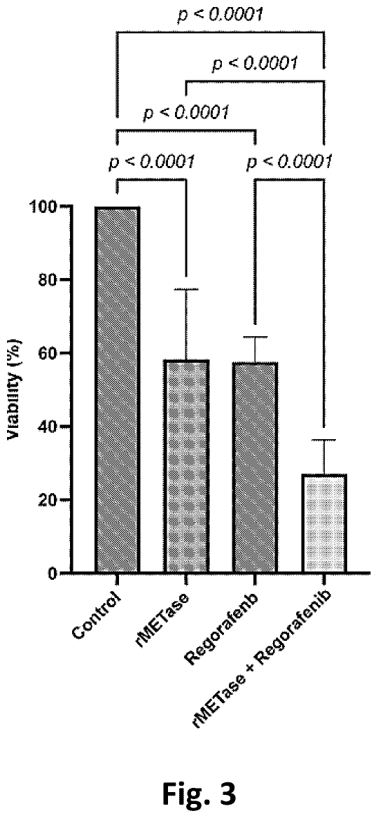

Determination of the IC₅₀ of rMETase alone and regorafenib alone and efficacy of their combination on HCT-116 cells in vitro. First, the sensitivity to rMETase alone and regorafenib alone of HCT-116 cells in vitro was evaluated, and IC₅₀ values were calculated. The IC₅₀ of rMETase on HCT-116 cells was found to be 0.61 U/ml, and the IC₅₀ of regorafenib was 2.26 μM (FIG. 1 and FIG. 2). The combination of rMETase and regorafenib at their IC₅₀ synergistically reduced the viability of HCT-116 cells in comparison to either agent alone at its IC₅₀ (FIG. 3).

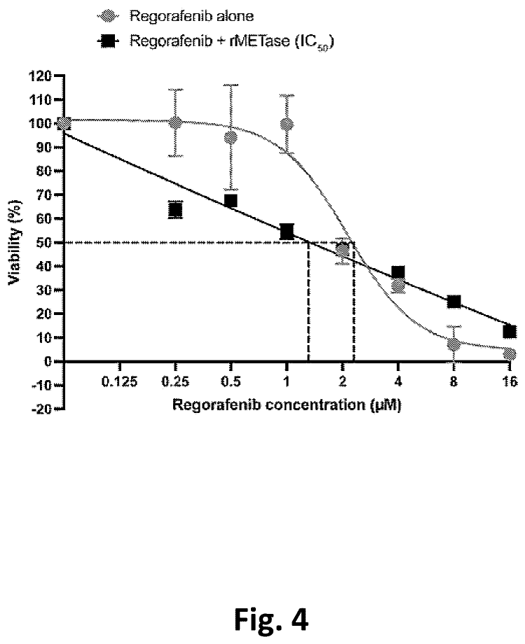

Referring to FIG. 4, while both recombinant methioninase alone (IC₅₀=0.61 U/ml) and regorafenib alone (IC₅₀=2.26 U/ml) inhibited the viability of HCT-116 cells, the combination of the recombinant methioninase and regorafenib was more than twice as effective as either alone. Addition of rMETase at 0.61 U/ml lowered the IC₅₀ of regorafenib from 2.26 μM to 1.46 μM. Thus, rMETase and regorafenib are synergistic, giving rise to the possibility of lowering the effective dose of regorafenib in patients, thereby reducing its severe toxicity, allowing more cancer patients to be treated with regorafenib.

The disclosed combination of rMETase and regorafenib can be used in the treatment of various kinds of cancers. This includes cancers for which regorafenib has already been approved. The combination may be used in the treatment of other types of cancer due to the reduced toxicity of regorafenib and synergetic action. The disclosed combination of rMETase and regorafenib may result in direct and/or indirect anti-cancer activity. The disclosed combination of rMETase and regorafenib may be introduced into the body of living humans or animals through a variety of suitable routes of administration. Preferably, the disclosed combination of rMETase and regorafenib can be administered through the oral route. It is understood that the route of administration may depend upon a variety of factors, such as the age and medical condition of a patient. Also, the targeted delivery routes to the tumors may be within the scope of the present invention. The combination may be administered orally as a single dose, for example, a tablet or capsule containing the combination of rMETase and regorafenib. The formulations of rMETase and regorafenib may also be administered separately to the patient, both orally. Also, the formulations of rMETase and regorafenib may be administered orally together at one time, or there may be a gap between the rMETase and regorafenib, and this gap may be of a predefined duration. This predefined duration may be clinically determined. It is obvious that the dosage regimen can be clinically determined and may depend upon a variety of factors. Any suitable dosage regimen and any suitable formulations of the rMETase and regorafenib are within the scope of the present invention.

Also disclosed is a composition comprising rMETase and regorafenib. Also, disclosed is a formulation comprising the rMETase and regorafenib. Also, disclosed is a single dosage unit comprising rMETase and regorafenib. The composition, the formulation, or the single dosage form may allow oral administration of the rMETase and regorafenib to a patient. The composition may include suitable pharmaceutically acceptable excipients for formulating the dosage forms by known pharmaceutical procedures. It is obvious that the choice of pharmaceutically acceptable excipients may depend upon the dosage form. For example, tablets, capsules, and powders may be prepared by the conventional method using pharmaceutical-acceptable excipients, such as binding agents (e.g., starch, polyvinylpyrrolidone, cellulose), fillers (e.g., lactose and microcrystalline cellulose), disintegrating agents (e.g., croscarmellose sodium, crospovidone, and sodium starch glycolate), lubricants (e.g., magnesium stearate), and the like known to a skilled person for formulating oral dosage forms.

The disclosed composition containing the rMETase and regorafenib can be administered in suitable doses for the treatment of cancer in humans and animals. It is obvious that the amount of rMETase and regorafenib to be administered can be clinically determined and may depend upon the age and weight of the patient. The patient herein refers to a person or animal suffering from some form of cancer and requiring treatment. Also, the dose may depend upon the type of cancer to be treated and the route of administration. In determining the correct dosage, in vivo, in vitro, as well as computational studies can be conducted.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method for treatment of cancer in a patient suffering from the cancer, the method comprises:
administering regorafenib, or a hydrate, an isomer, a solvate, a metabolite, a pharmaceutically acceptable salt, or a prodrug thereof, and recombinant methioninase in effective amounts to the patient.

2. The method of claim 1, wherein the administration is through an oral route.

3. The method of claim 1, wherein the cancer is metastatic colorectal cancer.

4. The method of claim 1, wherein the cancer is advanced gastrointestinal stromal tumors.

5. The method of claim 1, wherein the cancer is advanced hepatocellular carcinoma.

6. A composition for the treatment of cancer, the composition comprises:
regorafenib, or a hydrate, an isomer, a solvate, a metabolite, a pharmaceutically acceptable salt, or a prodrug thereof;
and recombinant methioninase.

7. The composition of claim 6, wherein the composition is for oral administration.

8. An oral dosage form for the treatment of cancer, the oral dosage form comprising:
regorafenib, or a hydrate, an isomer, a solvate, a metabolite, a pharmaceutically acceptable salt, or a prodrug thereof;
recombinant methioninase, and
pharmaceutically acceptable excipients.

9. The oral dosage form of claim 8, wherein the oral dosage form is a tablet.

10. The oral dosage form of claim 8, wherein the oral dosage form is a capsule.
